(19) United States
(12) Patent Application Publication
(10) Pub. No.: US 2021/0260348 A1
(43) Pub. Date: Aug. 26, 2021

(54) MEDICAL DEVICE AND MEDICAL DEVICE SET

(71) Applicant: TERUMO KABUSHIKI KAISHA, Tokyo (JP)

(72) Inventors: Mitsunori YOSHIKAWA, Tokyo (JP); Yoshiyuki SAITO, Tokyo (JP); Tetsuya FUKUOKA, Kanagawa (JP)

(73) Assignee: TERUMO KABUSHIKI KAISHA, Tokyo (JP)

(21) Appl. No.: 17/171,478

(22) Filed: Feb. 9, 2021

(30) Foreign Application Priority Data
Feb. 26, 2020 (JP) 2020-030626

(51) Int. Cl. A61M 25/10

(52) U.S. Cl. CPC A61M 25/10181; A61M 2025/105; A61M 2025/1063; A61M 2025/1065

(57) ABSTRACT

A medical device and a medical device set are disclosed capable of improving passability of a balloon catheter through a body lumen and protection of a balloon. The medical device is an auxiliary device for the balloon catheter, and the medical device has an elongated base shaft, and a distal end member including an extending portion that extends along a longitudinal direction of the base shaft and a balloon protecting portion that is provided on a distal end side relative to the extending portion and is formed in a spiral shape. The balloon protecting portion includes a space portion configured to at least partially accommodate the balloon of the balloon catheter that is provided inward in a radial direction of the spiral shape, and a tapered portion having an outer diameter which decreases toward the distal end side.

### CROSS-REFERENCES TO RELATED APPLICATIONS

This application claims priority to Japanese Application No. 2020-030626 filed on Feb. 26, 2020, the entire content of which is incorporated herein by reference.

### FIELD OF THE INVENTION

The present disclosure generally relates to a medical device and a medical device set.

### BACKGROUND DISCUSSION

A balloon catheter is a medical instrument that expands a lesion area (stenosed site, etc.) formed in a body lumen such as blood vessel (see, for example, Japanese Patent Application Publication No. 2013-192755).

The balloon catheter includes an elongated shaft and a balloon disposed at a distal end portion of the shaft.

The balloon catheter is introduced into the body lumen from a puncture site via a guiding catheter, etc., and is delivered to the lesion area. The balloon catheter can perform treatment on a blood vessel wall or the like by inflating the balloon at the lesion area.

However, when the balloon catheter is delivered to the lesion area (at the time of delivery), the following problems may occur, for example.

The body lumen into which the balloon catheter is inserted may not be flat and may be curved or partially narrowed. As a result, it may be difficult for the balloon catheter to move (pass) inside the body lumen. Therefore, it is necessary to improve the passability (pushability) of the balloon catheter through the body lumen.

Moreover, during delivery, an outer surface of the balloon of the balloon catheter may hit the wall surface of the body lumen. When the balloon hits the wall surface of the body lumen, the coating on the outer surface may peel off. Therefore, it is necessary to improve the protection of the balloon against the body lumen.

### SUMMARY

A medical device and a medical device set are disclosed for a balloon catheter capable of improving the passability of the balloon catheter through a body lumen and the protection of a balloon.

A medical device according to one aspect of the disclosure is an auxiliary device for a balloon catheter, and the medical device includes an elongated base shaft; a distal end member, the distal end member including an extending portion that extends along a longitudinal direction of the base shaft, and a protecting portion that is provided on a distal end side of the extending portion and is formed in a spiral shape; the protecting portion including a space portion that is provided inward in a radial direction of the spiral shape, and wherein the space portion is configured to at least partially accommodate a balloon of the balloon catheter; and a tapered portion on a distal end of the protecting portion, the tapered portion having an outer diameter which decreases toward the distal end side of the distal end member.

According to another aspect, a medical device set is disclosed, which includes a balloon catheter having a balloon configured to be inflated and deflated by supplying or discharging a fluid; and a medical device including an elongated base shaft, and a distal end member provided on a distal end side of the base shaft, the distal end member including an extending portion that extends along a longitudinal direction of the base shaft, and a protecting portion that is provided on a distal end side relative to the extending portion and is formed in a spiral shape, the protecting portion including a space portion being provided inward in a radial direction of the spiral shape, the space being configured to at least partially accommodate the balloon of the balloon catheter, and a tapered portion on a distal end of the protecting portion, the tapered portion having an outer diameter which decreases toward the distal end side of the distal end member.

According to a further aspect, a method is disclosed for applying a drug in a body lumen, the method comprising: assembling a medical device to a balloon catheter, the medical device including an elongated base shaft and a distal end member provided on a distal end side of the base shaft, the distal end member formed in a spiral shape having a space portion being provided inward in a radial direction of the spiral shape and disposing a drug-coated balloon of the balloon catheter in the space; introducing the medical device assembled to the balloon catheter into the body lumen; moving the medical device in a proximal direction relative to the drug-coated balloon of the balloon catheter; inflating the drug-coated balloon of the balloon catheter into a lesion area; and applying a drug from the drug-coated balloon to the lesion area.

According to the medical device and the medical device set, the passability of the balloon catheter through the body lumen and the protection of the balloon are improved.

### BRIEF DESCRIPTION OF THE DRAWINGS

FIG. 1 is a perspective view of a medical device set according to an exemplary embodiment.

FIG. 2 is a side view of the medical device set according to an exemplary embodiment.

FIG. 3 is a partial cross-sectional view of a protecting portion of a medical device according to the exemplary embodiment.

FIG. 4 is an exploded perspective view of the medical device set according to the exemplary embodiment.

FIG. 5 is a flow chart showing a treatment method according to the exemplary embodiment.

FIG. 6A is a diagram showing a state in which the medical device set has been inserted into a body lumen in accordance with an exemplary embodiment.

FIG. 6B is a diagram showing a state in which a balloon is exposed relative to the body lumen by moving a protecting portion covering the balloon of a balloon catheter in accordance with the exemplary embodiment.

FIG. 6C is a diagram showing a state in which the balloon is inflated in accordance with the exemplary embodiment.

### DETAILED DESCRIPTION

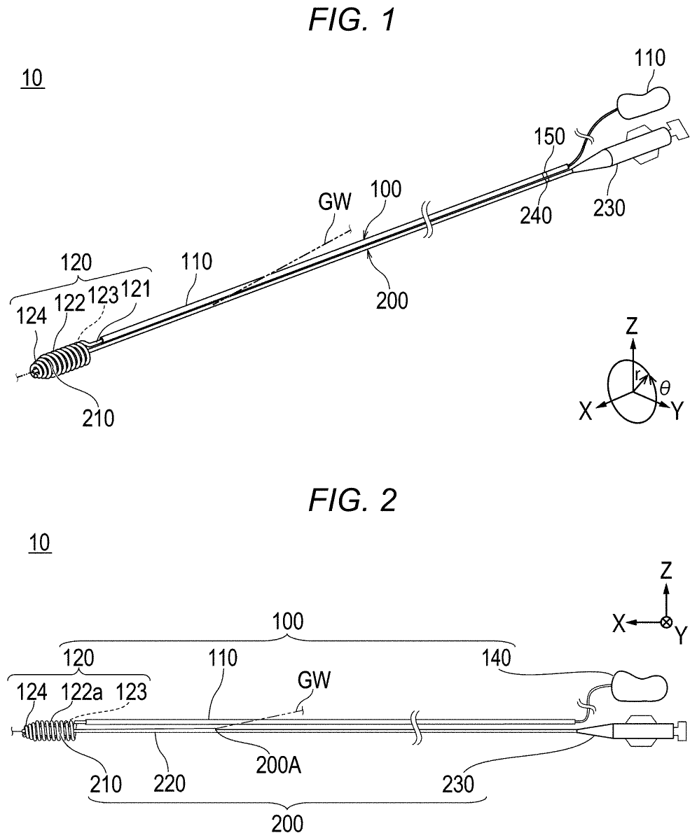

Set forth below with reference to the accompanying drawings is a detailed description of embodiments of a medical device and a medical device set representing examples of the inventive medical device and medical device set. Note that since embodiments described below are preferred specific examples of the present disclosure, although various technically preferable limitations are given, the scope of the present disclosure is not limited to the embodiments unless otherwise specified in the following descriptions. It should be noted that dimensional ratios in the drawings may be exaggerated for convenience of explanation and may differ from the actual ratios. Further, hereinafter, the longitudinal direction of a base shaft 110 is referred to as a longitudinal direction X. In addition, the circumferential direction of the base shaft 110 that intersects the longitudinal direction X is referred to as a circumferential direction θ, and the radial direction of the base shaft 110 with respect to the circumferential direction θ is referred to as a radial direction r. FIGS. 1 to 6C are diagrams for explaining a medical device set 10 according to a first exemplary embodiment.

As shown in FIGS. 1 and 4, the medical device set 10 according to the present exemplary embodiment includes a medical device 100 and a balloon catheter 200.

In the present disclosure, the balloon catheter 200 will be described as a drug-coated balloon (DCB) for applying a drug to a lesion area L of a blood vessel B (equivalent to a “body lumen”). When the drug-coated balloon is delivered to the lesion area L, the drug applied to a balloon 210 may flow out as the balloon 210 hits the wall surface of the blood vessel B in front of the lesion area L. Therefore, in order to effectively apply the drug to the lesion area L, it is necessary to protect the balloon 210 during delivery. The body lumen to which the drug is applied is not limited to a blood vessel, and may be, for example, a bile duct, trachea, esophagus, other gastrointestinal tracts, urethra, aural and nasal cavities, etc., preferably a coronary artery or an artery of lower extremity.

Medical Device

The medical device 100 is an auxiliary device for the balloon catheter 200.

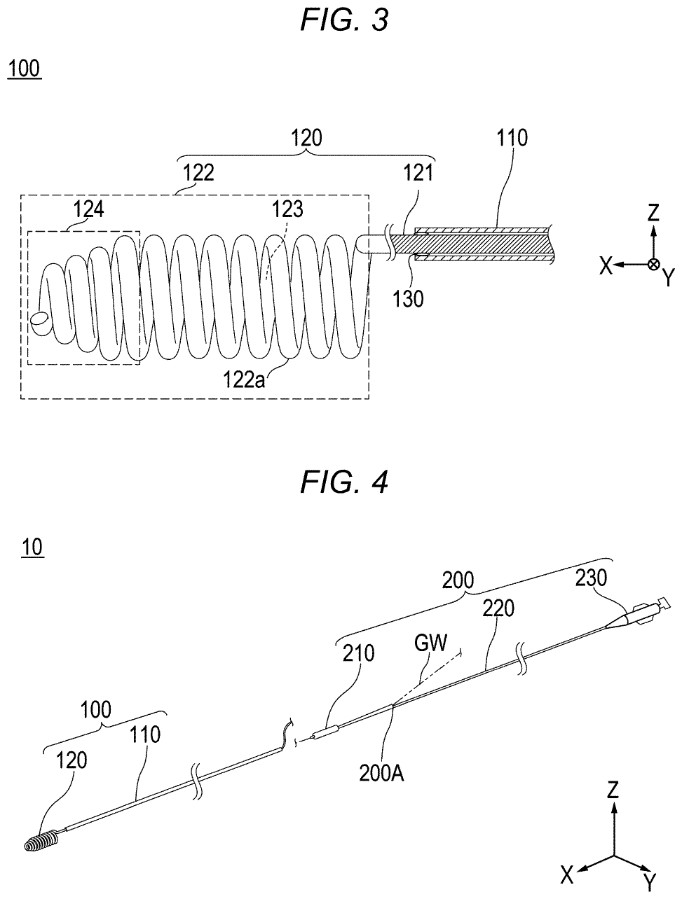

As shown in FIGS. 2 and 3, the medical device 100 includes the base shaft 110, a distal end member 120, and a fixing portion 130. In the description of the specification, the side of the medical device 100 that is introduced into a living body (the distal end member 120 side) is referred to as a “distal end side”, the base shaft 110 side is referred to as a “base end side” or “proximal end side”, and the direction in which the base shaft 110 extends (the horizontal direction in FIG. 2) is referred to as an axial direction or the longitudinal direction X.

The base shaft 110 is elongated and hollow. The base shaft 110 includes a member having a predetermined rigidity, and is configured to be capable of being inserted into a body lumen. That is, the base shaft 110 is configured to be capable of deforming in accordance with the shape of the body lumen while extending in a substantially linear shape when no external force is applied. The base shaft 110 is not limited as long as the base shaft 110 can rather easily transmit an operation at hand to the distal end side and can deform according to the body lumen. For example, the base shaft 110 may be made with a metal or an alloy, such as a super-elastic alloy, for example, nickel and titanium (Ni—Ti) alloy, brass, stainless steel (SUS), or aluminum.

In accordance with an exemplary embodiment, the distal end member 120 is a wire. The distal end member 120 is provided at least partially on the distal end side of the base shaft 110, and similar to the base shaft 110, the distal end member 120 can rather easily transmit an operation at hand to the distal end side and can deform according to the body lumen. In the exemplary embodiment, the distal end member 120 is made of the same material as that of the base shaft 110. However, the material for the distal end member 120 and the material for the base shaft 110 may be different.

As shown in FIG. 3, the distal end member 120 includes an extending portion 121 that extends along the longitudinal direction X of the base shaft 110, and a balloon protecting portion (equivalent to a “protecting portion”) 122 that is provided on the distal end side of (i.e., relative to) the extending portion 121.

In accordance with an exemplary embodiment, the extending portion 121 has a substantially linear shape. As shown in FIG. 3, the base end side (i.e., proximal end side) of the extending portion 121 is at least partially inserted into a lumen of the base shaft 110 from the distal end side of the base shaft 110 toward the base end side of the base shaft 110, and the distal end side of the extending portion 121 is disposed so as to partially extend toward the distal end side of the base shaft 110. Therefore, the extending portion 121 can improve the rigidity of the base end side of the medical device 100. As a result, the distal end member 120 can improve the passability of the medical device 100 through the body lumen.

In accordance with an exemplary embodiment, the rigidity of the medical device 100 according to the embodiment increases from the distal end side toward the base end side, and thus it is possible to suppress the occurrence of a sudden physical property difference in each part in the longitudinal direction X of the medical device 100. As a result, it is possible to suppress the occurrence of kinks and the like on the distal end side of the medical device 100.

In accordance with an exemplary embodiment, the balloon protecting portion 122 has a spiral shape. The balloon protecting portion 122 is provided on the distal end side relative to the base shaft 110, and is formed in a spiral shape in the longitudinal direction X of the extending portion 121 (or the base shaft 110).

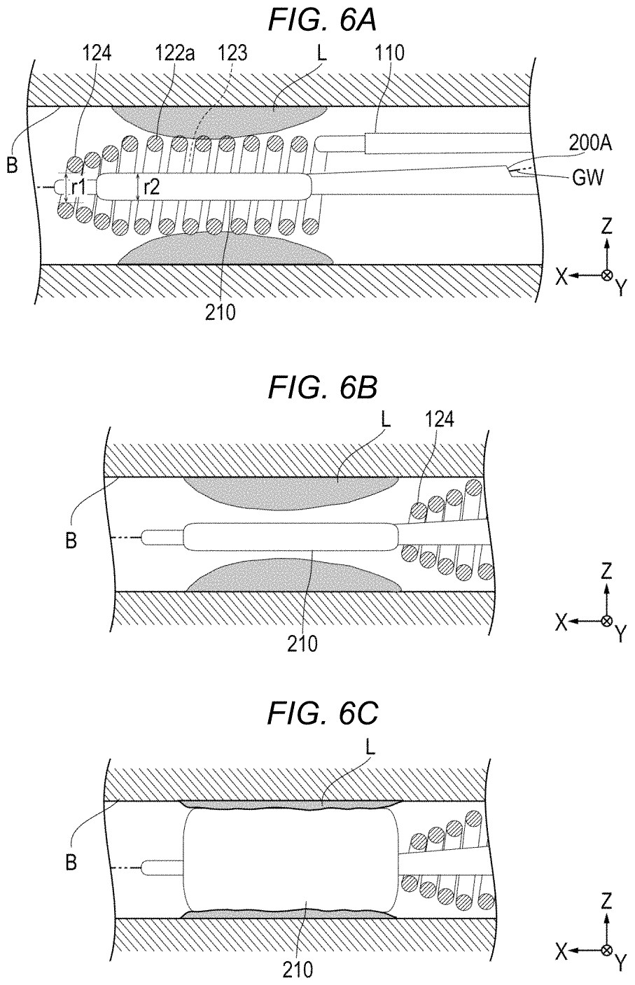

The balloon protecting portion 122 has a space portion 123 which is provided inward relative to a wire 122a forming the balloon protecting portion 122 in the radial direction r of the spiral shape (see FIG. 6A). Therefore, when the balloon catheter 200 is assembled to the medical device 100, the balloon 210 is at least partially accommodated in the space portion 123 formed by the balloon protecting portion 122. Thus, when delivering the balloon catheter 200 to the lesion area L of the blood vessel B in a state in which the balloon catheter 200 has been assembled to the medical device 100, the balloon protecting portion 122 can help prevent the balloon 210 from hitting the wall surface of the blood vessel B in front of the lesion area L. As a result, the balloon protecting portion 122 can improve the protection of the balloon 210 against the body lumen.

As shown, for example, in FIG. 6A, the balloon protecting portion 122 has a tapered portion 124 having an outer diameter which at least partially decreases (the outer diameter gradually decreases) toward the distal end side of the distal end member 120 (the balloon protecting portion 122). The tapered portion 124 can improve the insertion of the medical device 100 into the body lumen.

In accordance with an exemplary embodiment, a minimum inner diameter r1 of the tapered portion 124 (the balloon protecting portion 122) is larger than a maximum outer diameter r2 of the balloon 210 of the balloon catheter 200 assembled to the medical device 100 (see FIG. 6A). The outer diameter r2 of the balloon 210 refers to an outer diameter in a state before inflation. With such a configuration, when the balloon catheter 200 is assembled to the medical device 100, a gap is formed between the balloon 210 and the wire 122a forming the balloon protecting portion 122. As a result, the balloon protecting portion 122 can help prevent the balloon 210 from hitting the wall surface of the blood vessel B during delivery of the balloon catheter 200.

The length of the balloon protecting portion 122 in the longitudinal direction X is not limited as long as the balloon protecting portion 122 can partially accommodate and protect the balloon 210 of the balloon catheter 200 assembled to the medical device 100. However, the length is preferably at least greater than the entire length of the balloon 210. Moreover, the type of wire of the wire 122a forming the balloon protecting portion 122, the pitch and the number of turns of the wire 122a extending in a spiral direction are not limited. In addition, the ratio of the tapered portion 124 to the length of the balloon protecting portion 122 in the longitudinal direction X is not limited.

As shown in FIG. 3, the fixing portion 130 (i.e., a weld or an adhesive) fixes the position of the distal end member 120 with respect to the base shaft 110. As a result, the fixing portion 130 can fix the position of the balloon protecting portion 122 with respect to the base shaft 110. As shown in FIG. 3, the fixing portion 130 is provided on the distal end side of the base shaft 110. As described above, since the distal end member 120 is at least partially inserted into the lumen of the base shaft 110 from the distal end side to the base end side of the base shaft 110, the fixing portion 130 can fix the base shaft 110 and the distal end member 120 in a state in which the base shaft 110 and the distal end member 120 are overlapped. As a result, a connection portion between the base shaft 110 and the distal end member 120 can be reinforced, and the occurrence of kinks and the like can be suppressed.

As a fixing method for the fixing portion 130, for example, welding such as brazing and soldering may be applied. For a fixation point, full-circled welding or spot welding may be performed on materials forming the base shaft 110 and the distal end member 120. The fixing method is not limited to welding, and, for example, adhesion with an adhesive may also be applied.

The base shaft 110 and the distal end member 120 have been described above as separate parts. However, the base shaft 110 and the distal end member 120 may be integrally formed, and a part of a wire having the same outer diameter may be coiled to form the distal end member 120. Alternatively, the outer diameter of a part of the wire may be reduced, and the wire may be coiled to form the distal end member 120 while the part of the wire having a large outer diameter may be used as the base shaft 110.

Further, the base shaft 110 may be a solid elongated body.

Moreover, the cross-sectional shape of the base shaft 110 or the distal end member 120 is not limited. The cross-sectional shape of the base shaft 110 and the distal end member 120 is preferably a substantially perfect circle (or an annular shape); however, the cross-sectional shape of base shaft 110 and the distal end member 120 may be, for example, an elliptical shape, a rectangular shape, or other polygonal shapes.

Further, the base shaft 110 and the distal end member 120 may be coated with a hydrophilic coating. The material for the hydrophilic coating may be any material as long as it absorbs water and exhibits a swelling property. For example, hydrophilic materials may be used for the material of the hydrophilic coating. Examples of the hydrophilic materials can include polyvinylpyrrolidone, polyvinyl alcohol, polyethylene oxide-based polymer, cellulose-based polymer, acrylamide-based polymer, hyaluronic acid, polyacrylic acid, maleic anhydride-based polymer, water-soluble nylon, and derivatives of the hydrophilic materials disclosed above.

In addition, the medical device 100 may further include a hand portion 140. The hand portion 140 may be provided on the base end side (i.e., proximal end side) of the base shaft 110 and may be configured to be grasped by a person who performs a procedure such as a doctor. The hand portion 140 may be formed of a relatively rigid member such as plastic, and may be a hub or in a flat plate shape.

Balloon Catheter

In accordance with an exemplary embodiment, the balloon catheter 200 is configured with a rapid exchange type balloon catheter generally used in the medical field.

As shown in FIGS. 2 and 4, the balloon catheter 200 includes the balloon 210 that can be inflated and deflated by supplying or discharging a fluid, an elongated shaft 220 where the balloon 210 is disposed at a distal end portion, and a hub 230 disposed on a base end side of the shaft 220. The shaft 220 may be provided with a guide wire port 200A to introduce a guide wire GW into a lumen of the shaft 220.

As shown in FIG. 6A, the balloon catheter 200 can be at least partially inserted into the space portion 123 formed by the balloon protecting portion 122 of the medical device 100.

The balloon catheter 200 according to the exemplary embodiment may be delivered inside the blood vessel B while being inserted into the space portion 123. Therefore, an outer surface or the like of the balloon 210 can be prevented from being damaged as the balloon 210 hits the wall surface of the blood vessel B. In accordance with an exemplary embodiment, a drug carried by the balloon catheter 200 is preferably a compound that suppresses the proliferation of vascular endothelial cells such as an anticancer drug and an immunosuppressant, and specifically, for example, the drug carried by the balloon catheter 200 may include a compound containing at least one or more selected from the group consisting of paclitaxel, sirolimus, everolimus and biolimus, or a mixture paclitaxel, sirolimus, everolimus and/or biolimus. In accordance with an exemplary embodiment, the drug is preferably solid, and more preferably crystalline.

A marker 240 (see FIG. 1) or the like for aligning the balloon 210 with the lesion area L of the blood vessel B or with the medical device 100 may be provided in the vicinity of the hand of the balloon catheter 200 (near the base end portion or proximal end portion). When the marker 240 is used for alignment with the medical device 100, a marker 150 corresponding to the marker 240 may be provided in the medical device 100 (see FIG. 1).

Treatment Method

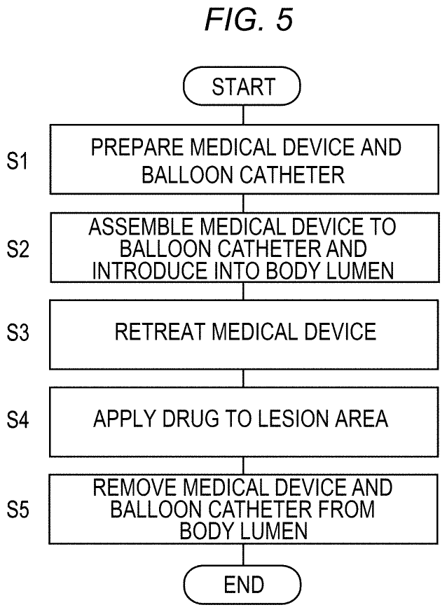

Next, a method for applying a drug according to the embodiment will be described with reference to FIGS. 5 to 6C.

The method of applying the drug includes, when outlined with reference to FIG. 5, preparing the medical device set 10, (S1), introducing the medical device set 10 into a body lumen (S2), retreating the medical device 100 (S3), applying a drug to the lesion area L (S4), and removing the medical device set 10 (S5).

First, an operator such as a doctor prepares the balloon catheter 200 before starting a procedure of applying a drug. In addition, the operator prepares the medical device 100 used to protect the balloon 210 of the balloon catheter 200 (51).

Next, as shown in FIGS. 2 and 4, the operator prepares the medical device set 10 by assembling the medical device 100 to the balloon catheter 200 (disposing the balloon 210 in the space portion 123 of the balloon protecting portion 122) and introduces the medical device set 10 into the lesion area L of an artery, which is an example of a body lumen (S2). Specifically, the operator introduces the medical device set 10, for example, from a radial artery of an arm, and disposes the balloon protecting portion 122 on the lesion area L, which is a stenosed site or an occluded part of a coronary artery. As described above, the balloon protecting portion 122 is made with a wire. Therefore, the balloon 210 can pass through the lesion area L which may be a stenosed site or an occluded part while flexibly deforming according to an operation of the operator, and is rather easily disposed on the lesion area L. When introducing the medical device set 10 into the blood vessel B, the operator can appropriately use a medical instrument such as a guiding catheter.

Next, as shown in FIG. 6A, the operator disposes the balloon 210 of the balloon catheter 200 on the lesion area L (a site to be applied) of the blood vessel B. Then, as shown in FIG. 6B, the operator retreats the medical device 100 toward the base end side so that the distal end side of the balloon protecting portion 122 of the medical device 100 is disposed closer to the base end side than the balloon 210 of the balloon catheter 200 (S3). Since the balloon protecting portion 122 is made with a wire as described above, the balloon protecting portion 122 moves to the base end side of the medical device set 10 while flexibly deforming according to the operation of the operator.

Then, as shown in FIG. 6C, the operator inflates the balloon 210. As a result, the balloon 210 is displaced outward in the radial direction r as shown in FIG. 6C, and the outer surface adheres to the inner wall surface of the lesion area L. Therefore, the drug carried by the balloon 210 is applied to the inner wall surface of the lesion area L (S4).

The number of times that the balloon 210 is inflated is not limited as long as the number of times that the balloon 210 is inflated is one or more.

Next, the operator deflates the balloon 210 (not shown). After deflating the balloon 210, the operator removes the medical device set 10 from the body lumen by moving the medical device set 10 to the base end side (S5). For the retreat of the medical device set 10, the medical device 100 may be removed together with the balloon catheter 200, or the medical device 100 may be removed outside the body before applying the drug to the lesion area L.

As described above, the medical device 100 according to the exemplary embodiment is an auxiliary device for the balloon catheter 200, and includes the elongated base shaft 110, and the distal end member 120 including the extending portion 121 that extends along the longitudinal direction X of the base shaft 110, and the balloon protecting portion (the protecting portion) 122 that is provided on the distal end side relative to the extending portion 121 and is formed in a spiral shape. In accordance with an exemplary embodiment, the balloon protecting portion 122 is provided inward in the radial direction r of the spiral shape, and includes the space portion 123 that can at least partially accommodate the balloon 210 of the balloon catheter 200, and the tapered portion 124 having an outer diameter which decreases toward the distal end side.

With this configuration, when the balloon catheter 200 is delivered in a state in which the balloon catheter 200 is assembled to the medical device 100, the balloon protecting portion 122 can prevent the balloon 210 from hitting the wall surface of the blood vessel B, and thus the balloon 210 can be protected against the body lumen. Moreover, the balloon protecting portion 122 can improve the insertion of the balloon catheter 200 into the body lumen, particularly the passability through the lesion area L, by utilizing the tapered portion 124.

Further, the minimum inner diameter r1 of the tapered portion 124 is larger than the maximum outer diameter r2 of the balloon 210 of the balloon catheter 200. In this way, when the balloon catheter 200 is assembled to the medical device 100, a gap is formed between the balloon 210 and the wire 122a forming the balloon protecting portion 122. As a result, when the balloon catheter 200 is delivered in a state in which the balloon catheter 200 is assembled to the medical device 100, the balloon protecting portion 122 can prevent the balloon 210 from hitting the wall surface of the blood vessel B.

Moreover, the base shaft 110 is hollow, and the base end side of the extending portion 121 is at least partially inserted into the lumen of the base shaft 110 from the distal end side to the base end side of the base shaft 110. As a result, the extending portion 121 can improve the rigidity of the base end side of the medical device 100. Therefore, the distal end member 120 can improve the passability of the medical device 100 through the body lumen, for example, the passability through the lesion area L.

In addition, the medical device 100 includes the fixing portion 130 that is provided on the distal end side of the base shaft 110 and fixes the position of the balloon protecting portion 122 with respect to the base shaft 110. As a result, the position of the distal end member 120 with respect to the base shaft 110 can be fixed.

Further, the medical device set 10 according to the embodiment includes the balloon catheter 200 having the balloon 210 that can be inflated and deflated by supplying or discharging a fluid, and the medical device 100 including the elongated base shaft 110, and the distal end member 120 provided on the distal end side of the base shaft 110. The distal end member 120 includes the extending portion 121 that extends along the longitudinal direction X of the base shaft 10, and the balloon protecting portion 122 that is provided on the distal end side relative to the extending portion 121 and is formed in a spiral shape. The balloon protecting portion 122 is provided inward in the radial direction r of the spiral shape, and includes the space portion 123 that can at least partially accommodate the balloon 210 of the balloon catheter 200, and the tapered portion 124 having an outer diameter which decreases toward the distal end side.

With this configuration, the balloon protecting portion 122 can help prevent the balloon 210 from hitting the wall surface of the blood vessel B during the delivery of the balloon catheter 200. As a result, the protection of the balloon 210 against the body lumen can be improved. In addition, the balloon protecting portion 122 can improve the insertion of the balloon catheter 200 into the body lumen by utilizing the tapered portion 124.

It should be noted that the disclosure is not limited to the aforementioned embodiment, and can be variously modified within the scope of the claims.

For example, the fixing portion 130 of the medical device 100 may be configured by a member that allows the operator to select whether to fix the position of the distal end member 120 (the balloon protecting portion 122) with respect to the base shaft 110. The distal end member 120 may be configured to be movable in the lumen of the base shaft 110.

Moreover, the type of the balloon catheter 200 is not limited. The balloon catheter 200 may be, for example, a drug-eluting balloon catheter that applies a drug to the lesion area L by releasing the drug from a micropore formed in the balloon 210.

In addition, the balloon catheter 200 has been described as a rapid exchange type balloon catheter. However, the balloon catheter 200 may be, for example, an over-the-wire type balloon catheter.

The detailed description above describes embodiments of a medical device and medical device set. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device which is an auxiliary device for a balloon catheter, the medical device comprising:
an elongated base shaft;
a distal end member, the distal end member including an extending portion that extends along a longitudinal direction of the base shaft, and a protecting portion that is provided on a distal end side of the extending portion and is formed in a spiral shape;
the protecting portion including a space portion that is provided inward in a radial direction of the spiral shape, and wherein the space portion is configured to at least partially accommodate a balloon of the balloon catheter; and
a tapered portion on a distal end of the protecting portion, the tapered portion having an outer diameter which decreases toward the distal end side of the distal end member.

2. The medical device according to claim 1, wherein the tapered portion has a minimum inner diameter which is larger than a maximum outer diameter of the balloon of the balloon catheter.

3. The medical device according to claim 1, wherein the base shaft is hollow; and
a proximal end side of the extending portion is at least partially inserted into a lumen of the base shaft from a distal end side of the base shaft toward a proximal end side of the base shaft.

4. The medical device according to claim 1, further comprising:
a fixing portion that is provided on the distal end side of the base shaft and fixes a position of the protecting portion with respect to the base shaft.

5. The medical device according to claim 4, wherein the fixing portion is a weld or an adhesive configured to fix the position of the protecting portion with respect to the base shaft.

6. The medical device according to claim 1, wherein the distal end member is a wire.

7. A medical device set, the medical device set comprising:
a balloon catheter having a balloon configured to be inflated and deflated by supplying or discharging a fluid; and
a medical device including an elongated base shaft, and a distal end member provided on a distal end side of the base shaft, the distal end member including an extending portion that extends along a longitudinal direction of the base shaft, and a protecting portion that is provided on a distal end side relative to the extending portion and is formed in a spiral shape, the protecting portion including a space portion being provided inward in a radial direction of the spiral shape, the space being configured to at least partially accommodate the balloon of the balloon catheter, and a tapered portion on a distal end of the protecting portion, the tapered portion having an outer diameter which decreases toward the distal end side of the distal end member.

8. The medical device set according to claim 7, wherein the tapered portion of the medical device has a minimum inner diameter which is larger than a maximum outer diameter of the balloon of the balloon catheter.

9. The medical device set according to claim 7, wherein the base shaft of the medical device is hollow; and
a proximal end side of the extending portion is at least partially inserted into a lumen of the base shaft from a distal end side of the base shaft toward a proximal end side of the base shaft.

10. The medical device set according to claim 7, further comprising:
a fixing portion that is provided on the distal end side of the base shaft and fixes a position of the protecting portion with respect to the base shaft.

11. The medical device set according to claim 10, wherein the fixing portion is a weld or an adhesive configured to fix the position of the protecting portion with respect to the base shaft.

12. The medical device set according to claim 7, wherein the distal end member of the medical device is a wire.

13. The medical device set according to claim 7, wherein a length of the protecting portion in a longitudinal direction is at least greater than a length of the balloon of the balloon catheter.

14. The medical device set according to claim 7, wherein the elongated shaft of the balloon catheter includes a guide wire port configured to introduce a guide wire into a lumen of the elongated shaft of the balloon catheter.

15. The medical device set according to claim 7, wherein the balloon of the balloon catheter is a drug-coated balloon.

16. A method for applying a drug in a body lumen, the method comprising:
assembling a medical device to a balloon catheter, the medical device including an elongated base shaft and a distal end member provided on a distal end side of the base shaft, the distal end member formed in a spiral shape having a space portion being provided inward in a radial direction of the spiral shape and disposing a drug-coated balloon of the balloon catheter in the space;
introducing the medical device assembled to the balloon catheter into the body lumen;
moving the medical device in a proximal direction relative to the drug-coated balloon of the balloon catheter;
inflating the drug-coated balloon of the balloon catheter into a lesion area; and
applying a drug from the drug-coated balloon to the lesion area.

17. The method according to claim 16, further comprising:
deflating the drug-coated balloon; and
removing the medical device and the balloon catheter from the body lumen.

18. The method according to claim 16, further comprising:
passing the balloon catheter through the lesion area;
displacing the drug-coated balloon outward in a radial direction;
adhering an outer surface of the drug-coated balloon to an inner wall surface of the lesion area; and
applying a drug of the drug-coated balloon to the inner wall surface of the lesion area.

19. The method according to claim 16, wherein the lesion area is a stenosed site or an occluded part of a coronary artery, the method further comprising:
inflating the drug-coated balloon of the balloon catheter one or more times within the lesion area.

20. The method according to claim 16, wherein a distal end of the spiral shape has a tapered portion, the tapered portion having an outer diameter which decreases toward the distal end side of the distal end member, the method further comprising:
removing the medical device outside of the body lumen before applying the drug of the drug-coated balloon to the lesion area.
